(19) United States
(12) Patent Application Publication
(10) Pub. No.: US 2021/0084953 A1
(43) Pub. Date: Mar. 25, 2021

(54) GUM ARABIC/CHITOSAN COACERVATE SYSTEM

(71) Applicant: Firmenich SA, Meyrin 2 (CH)

(72) Inventors: Amal Elabbadi, Geneva 8 (CH); Philipp Erni, Geneva 8 (CH)

(21) Appl. No.: 16/634,235

(22) PCT Filed: Jul. 24, 2018
(86) PCT No.: PCT/EP2018/070069

§ 371 Date: Jan. 27, 2020

(30) Foreign Application Priority Data
Jul. 27, 2017 (EP) 17183475.7

(51) Int. Cl. A23L 29/25; A23L 29/275; A23P 10/35; A23L 27/00; A23C 9/137; A23C 9/154; A23G 9/34; A23L 35/00; A23L 27/60; A23G 1/40

(52) U.S. Cl. CPC A23L 29/25; A23L 29/275; A23P 10/35; A23L 27/72; A23C 9/137; A23V 2002/00; A23G 9/34; A23L 35/20; A23L 27/66; A23G 1/40; A23C 9/154

(57) ABSTRACT

Described herein is the method of using a complex coacervate system including Gum Arabic and chitosan as lubricating agents. Also described herein are consumer products including the complex coacervate system.

### TECHNICAL FIELD

The present invention relates to the field of lubricating agents that can be used for example as a fat replacer for the partial or full replacement of fat in consumer products. More specifically, the present invention relates to the use of a complex coacervate system comprising gum Arabic and chitosan as a lubricating agent.

Consumer products comprising those coacervate systems are also objects of the present invention.

### BACKGROUND OF THE INVENTION

Lubricating agents are known to be substances introduced to reduce friction between two surfaces. They are used in many different applications. Among them, one can cite for example food applications.

Indeed, faced with awareness of adverse effects of excessive dietary fat intake, people are modifying their dietary habits and eating less fat.

Consequently, for several years, many lubricating agents playing the role of fat replacers have been developed to meet this need.

Fat replacers have been widely disclosed in the prior art. One may cite for example U.S. Pat. No. 5,952,007 disclosing the use of complex coacervates of two or more biopolymer materials as fat replacers in food and cosmetic application. However, the process disclosed in this document requires a heating step where the biopolymers must be dissolved in water at a temperature between 30 and 100° C. Moreover, some parameters such as pressure or shear rate must fulfill criteria during the process leading to a complex process.

There is therefore a need to provide a lubricating agent that could be used for example as a fat replacer that would be easy to prepare and that would provide good performance in terms of texture.

The present invention satisfies this need by providing a complex coacervate system made of a specific combination of two different biopolymers.

### SUMMARY OF THE INVENTION

It has now been found that, a Gum Arabic/chitosan coacervate system exhibits high lubricating properties and could be used as a lubricating agent in different applications. Indeed, for example, when introduced in a flavoured product, the coacervate system of the present invention can be used as a fat replacer to provide an oily texture.

A first object of the present invention is therefore the use of a complex coacervate system as a lubricating agent in a consumer product, said complex coacervate system comprising a first biopolymer and a second biopolymer, wherein the first biopolymer is gum Arabic and wherein the second biopolymer is chitosan.

Other objects of the present invention are consumer products comprising the coacervate systems as defined above.

### BRIEF DESCRIPTION OF THE DRAWINGS

FIG. 1 represents the friction coefficient as a function of the sliding speed (mm/s) for the isolated biopolymers (Gum Arabic and chitosan) and for the complex coacervate droplets slurry of the present invention.

FIG. 2 represents the friction coefficient as a function of the sliding speed (mm/s) for the coacervate droplets slurry of the present invention when incorporated in a fat-free yogurt.

FIG. 3 represents the friction coefficient as a function of the sliding speed (mm/s) for the coacervate microcapsules slurry of the present invention and for an emulsion.

FIG. 4 represents the friction coefficient as a function of the sliding speed (mm/s) for the coacervate droplets slurry of the present invention compared to gelatin/gum Arabic coacervates.

### DETAILED DESCRIPTION OF THE INVENTION

Unless stated otherwise, percentages (%) are meant to designate a percentage by weight of a composition.

The term “complex coacervate system” used in the present invention encompasses two different systems, namely the complex coacervate droplets slurry and/or the complex coacervate microcapsules slurry. Those two systems are linked by a common feature that is the nature of the two biopolymers forming the coacervate system.

By “complex coacervate droplets” it is meant that droplets are only made of biopolymers comprising gum Arabic and chitosan, preferably consisting of gum Arabic and chitosan.

By “complex coacervate microcapsules”, it is meant that complex coacervate microcapsules are made of an oil-based core comprising a hydrophobic active ingredient and a complex coacervate shell made of biopolymers comprising gum Arabic and chitosan, preferably consisting of gum Arabic and chitosan.

The complex coacervate system defined in the present invention can be used as a lubricating agent in different applications such as food applications, cosmetic applications or bio-medical applications.

According to a particular embodiment, the lubricating agent is a fat replacer in flavored products (food products).

As previously explained, a lubricating agent is a material that is able to decrease the friction coefficient between two surfaces (skin-skin contact for personal care products or tongue-palate for flavored product).

According to an embodiment, the friction coefficient of the complex coacervate material defined in the present invention between any two surfaces decreases by up to 85% as compared to the friction coefficient of pure water measured between the same two surfaces, wherein the two surfaces comprise a acrylonitrile butadiene rubber or a biological tissue.

### Complex Coacervate Droplets Slurry

According to an embodiment, the complex coacervate system is a complex coacervate droplets slurry containing at least one complex coacervate comprising the first biopolymer and the second biopolymer, namely gum Arabic and chitosan.

Methods for preparing complex coacervates are well-known from the skilled person in the art.

According to an embodiment, the complex coacervate droplets slurry is obtainable by a process comprising the step of mixing the first and the second biopolymer in an aqueous vehicle under conditions sufficient to form a suspension of complex coacervate droplets, wherein this step is performed under acidic conditions.

To form the complex coacervate droplets slurry, gum Arabic and chitosan are mixed under specific temperature, pH and concentration conditions to induce polymer phase separation, so as to produce a suspension of complex coacervate droplets. The person skilled in the art will be able to select the optimal conditions (pH, ionic strength and temperature) according to the nature of those polyelectrolytes leading the desired complex coacervate formation.

According to the invention, the step of mixing is carried out under acidic conditions since chitosan needs to be dissolved in an acid such as acetic acid, lactic acid. Gum Arabic is typically dissolved in water at room temperature.

It has been found that the lubricating properties are optimal when the complex coacervate droplets slurry is obtainable by a process in which the pH is comprised between 2.5 and 5, preferably between 3 and 4.

Furthermore, according to this embodiment, the weight ratio between gum Arabic and chitosan is preferably comprised between 3 and 8, more preferably is equal to 4.

According to an embodiment, the total amount of the biopolymers is comprised between 1 w % and 10 w %, preferably between 2 w % and 8 w % based on the total weight of the slurry.

The complex coacervate droplets slurry can be submitted to a drying, like lyophilisation or spray-drying, to provide the coacervate droplets as such, i.e. in a powder form. It is understood that any standard method known by a person skilled in the art to perform such drying is applicable. In particular the slurry may be spray-dried preferably in the presence of a polymeric carrier material such as polyvinyl acetate, polyvinyl alcohol, dextrins, maltodextrin, natural or modified starch, sugars, vegetable gums such as gum acacia, pectins, xanthans, alginates, carrageenans or cellulose derivatives to provide microcapsules in a powder form. Preferably, the carrier is a gum Acacia. According to a particular embodiment, the carrier material contains free perfume oil or free flavour oil.

### Complex Coacervate Microcapsules Slurry

According to another embodiment, the complex coacervate system is a complex coacervate microcapsules slurry comprising at least one microcapsule having an oil-based core comprising a hydrophobic active ingredient, preferably a flavor or a perfume, and a coacervate shell made of the first biopolymer and the second biopolymer, namely gum Arabic and chitosan.

According to an embodiment, the complex coacervate microcapsules slurry is obtainable by a process comprising the steps of:
(i) mixing chitosan and Gum Arabic in an aqueous vehicle under conditions sufficient to form a suspension of complex coacervate droplets, wherein this step is performed under acidic conditions; and
(ii) adding a hydrophobic core material, preferably a flavor or a perfume, to the complex coacervates to form core/shell capsules each containing the core material encapsulated by a coacervate shell made of chitosan and Gum Arabic.

### Step (i): Mixing Gum Arabic and Chitosan in an Aqueous Vehicle

In a first step, Gum Arabic and chitosan are mixed under specific temperature, pH and concentration conditions to induce polymer phase separation, so as to produce a suspension of complex coacervate droplets. The person skilled in the art will be able to select the optimal conditions (pH, ionic strength and temperature) according to the nature of those polyelectrolytes leading to the desired complex coacervate formation.

According to the invention, the step of mixing is carried out under acidic conditions since chitosan needs to be dissolved in an acid such as acetic acid, lactic acid. Gum Arabic is typically dissolved in water at room temperature.

It has been found that the lubricating properties are optimal when the complex coacervate microcapsules slurry is obtainable by a process in which the pH is comprised between 2.5 and 5, preferably between 3 and 4.

According to this embodiment, the weight ratio between gum Arabic and chitosan is preferably comprised between 5 and 8, even more preferably is equal to 6.

According to an embodiment, the total amount of the biopolymers is comprised between 1 w % and 10 w %, preferably between 2 w % and 8 w % based on the total weight of the slurry.

### Step (ii): Adding a Hydrophobic Core Material to the Complex Coacervate Droplets

In step (ii), a hydrophobic core material, preferably a flavor or a perfume, is added to the complex coacervate droplets, wherein the complex coacervate droplets deposit as a coating layer around the core material active/solution interface to form core/shell capsules each containing the core material encapsulated by a coacervate shell made of chitosan and Gum Arabic. According to this embodiment, the coacervate shell is made of the complex coacervate droplets formed in step (i).

The hydrophobic core material can be added according to two different ways.

Indeed, in addition to the lubricating properties, it has been shown that the coacervate phase presents an efficient interfacial activity, which enables the stabilization of oil droplets.

Thus, according to an embodiment, an oil phase comprising the hydrophobic core material is added into the complex coacervate droplets under stirring wherein the complex coacervate droplets deposit as a coating layer around the core material active/solution interface to form core/shell capsules each containing the core material encapsulated by a coacervate shell made of chitosan and Gum Arabic.

According to another embodiment, an oil-in-water emulsion comprising the hydrophobic core material is added into the complex coacervate droplets wherein the complex coacervate droplets deposit as a coating layer around the emulsion comprising the core material active/solution interface to form core/shell capsules each containing the core material encapsulated by a coacervate shell made of chitosan and Gum Arabic.

According to this embodiment, the emulsion preferably comprises a stabilizer such as a protein.

Also, the coacervate has a suitable viscosity that allows the coacervate to deposit on the core material to form the capsule shell. The viscosity of the hybrid coacervate may be between 100 mPas and 4000 mPas at 20° C., and at shear rates that may be between 1/s and 100/s.

According to an embodiment, the active ingredient consists of a perfume or flavour. Alternative hydrophobic ingredients which could benefit from being encapsulated could be used either instead of a perfume or flavour, or in combination with a perfume or flavour. Non-limiting examples of such ingredients include a cosmetic, skin caring, malodour counteracting, bactericide, fungicide, pharmaceutical or agrochemical ingredient, a sanitizing agent, an insect repellent or attractant.

By “perfume oil” (or also “perfume”) or “flavour” what is meant here is an ingredient or composition that is a liquid at about 20° C. Said perfume or flavour oil can be a perfuming or flavouring ingredient alone or a mixture of ingredients in the form of a perfuming or flavouring composition. As a “perfuming ingredient” it is meant here a compound, which is used in perfuming preparations or compositions to impart as primary purpose a hedonic effect. In other words such an ingredient, to be considered as being a perfuming one, must be recognized by a person skilled in the art as being able to at least impart or modify in a positive or pleasant way the odor of a composition, and not just as having an odor. The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said ingredients may also be compounds known to release in a controlled manner various types of perfuming compounds.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

By “flavour ingredient or composition” it is meant here a flavouring ingredient or a mixture of flavouring ingredients, solvent or adjuvants of current use for the preparation of a flavouring formulation, i.e. a particular mixture of ingredients which is intended to be added to an edible composition or chewable product to impart, improve or modify its organoleptic properties, in particular its flavour and/or taste. Taste modulator as also encompassed in said definition. Flavouring ingredients are well known to a skilled person in the art and their nature does not warrant a detailed description here, which in any case would not be exhaustive, the skilled flavourist being able to select them on the basis of his general knowledge and according to the intended use or application and the organoleptic effect it is desired to achieve. Many of these flavouring ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor Ingredients, 1975, CRC Press or Synthetic Food Adjuncts, 1947, by M. B. Jacobs, can Nostrand Co., Inc. Solvents and adjuvants or current use for the preparation of a flavouring formulation are also well known in the art.

In a particular embodiment, the flavour is selected from the group consisting of terpenic flavours including citrus and mint oil, and sulfury flavours.

According to any one of the invention's embodiments, the oil represents between about 10% and 60% w/w, or even between 20% and 50% w/w, by weight, relative to the total weight of the dispersion of step (ii).

### Optional Steps

After the formation of the coacervate shell, the shell can be hardened using a cross-linking agent. Suitable agents for cross-linking include, but are not limited to, polyphosphate, genipin, formaldehyde, acetaldehyde, glutaraldehyde, glyoxal, chrome alum, and transglutaminase.

The complex coacervate microcapsules slurry can be submitted to a drying, like lyophilisation or spray-drying, to provide the microcapsules as such, i.e. in a powder form. It is understood that any standard method known by a person skilled in the art to perform such drying is applicable. In particular the slurry may be spray-dried preferably in the presence of a polymeric carrier material such as polyvinyl acetate, polyvinyl alcohol, dextrins, maltodextrin, natural or modified starch, sugars, vegetable gums such as gum acacia, pectins, xanthans, alginates, carrageenans or cellulose derivatives to provide microcapsules in a powder form. Preferably, the carrier is a maltodextrin. According to a particular embodiment, the carrier material contains free perfume oil or free flavour oil which can be same or different from the perfume or the flavour from the core of the microcapsules.

The coacervate system defined in the present invention is used in consumer products, preferably flavoured products or perfumed product.

According to a particular embodiment, the coacervate system defined in the present invention is used in a flavoured product. As flavored product, one may cite for example dairy products such as yoghurts and other fermented milk products, milk drinks, and cheese products, sauces, dressings.

According to a particular embodiment, the coacervate system defined in the present invention is used in a fragranced product. As fragranced product, one may cite for example lipsticks, creams, lotions such as sunscreen lotions.

Preferably the level of the coacervate systems defined in the present invention in the consumer products will be from 0.1 to 20% by weight of the composition, more preferred 0.2 to 10%, most preferred 0.5 to 5%.

### Consumer Products

Another object of the invention is a consumer product comprising the coacervate systems as defined in the present invention. Indeed, the coacervate systems can be used in different applications.

Flavoured Product

For example, they can be used in food and beverages, preferably comprising no amount or a limited amount of fat, where capsules made by coacervation are commonly used, including, but not limited to dairy products such as yoghurts and other fermented milk products, milk drinks, and cheese products, sauces, dressings.

Preferably the level of the coacervate systems defined in the present invention in the consumer products will be from 0.1 to 20% by weight of the composition, more preferred 0.2 to 10%, most preferred 0.5 to 5%.

Fragranced Product

The coacervate systems of the invention can also be used in perfumery applications where capsules made by coacervation can be used, including, but not limited to, lipsticks, creams, lotions such as sunscreen lotions.

### EXAMPLES

The following examples are provided as illustrations of the preferred embodiments of the invention and are not intended to limit the scope of the invention.

Tribology: Friction coefficients were measured by tribology in the coacervate phases, using the rheometer Physica (Anton Paar) mounted with a 3-pin on disc geometry (radius r=25 mm). In these measurements, the friction coefficient between two solid surfaces in relative motion and separated by the sample is measured. The 3-pin top plate rotates on a stationary disc, while exerting a constant normal force of 5N on the disc. Both parts of the device were coated with nitrile rubber as a model soft material

After transferring the sample on the lower part of the device (stationary part), the 3-pin geometry was lowered and brought into contact with the sample with a normal force of 3N. The sample was allowed to relax under the pressure for 3-5 minutes (we could notice a small decrease in the normal force from 3N to ˜2.5N) and the measurement was then run: the normal force was first increased and set to 5N before rotating the geometry at increasing speeds. The torque exerted during the rotation was measured and we could then calculate the friction coefficient μ according to the following formula:

μ=Mr/Nf

Where M (expressed in Nm) is the torque exerted by the instrument's engine to overcome the friction, r (m) is the radius of the 3-pin geometry, and Nf (N) is the normal force exerted on the sample.

All results given are averages from triplicate measurements.

A sample enabling a low friction coefficient is a lubricating material.

### Example 1

### Preparation of a Complex Coacervate Droplets Slurry Defined in the Invention

Gum arabic (GA) 20 wt % and chitosan (CTS) 2 wt % stock solutions were prepared respectively in deionized water and in acetic acid 1% by magnetic stirring.

The dissolution of CTS, which yields a very viscous solution, was helped by mild heating at 60° C.

Both solutions were stored in the fridge overnight before use to fully hydrate the biopolymers.

The amounts of CTS and GA solutions required to reach the wanted GA/CTS weight ratio were mixed in 20-ml vials with a magnetic stirrer (see table 1).

The pH was adjusted after mixing the biopolymers by adding dilute acetic acid and NaOH solutions. The suspensions were centrifuged 5 minutes at 1790 g (4000 rpm using centrifuge BR4i from Jouan) and the coacervate phase was separated from the equilibrium solution.

R=GA/CTS

T=% GA+% CTS

TABLE 1
Composition of different samples of
Gum Arabic/chitosan droplets slurry

| CTS | GA | R | T | Sample name |
|---|---|---|---|---|
| 0.5% | 2% | 4 | 2.5% | CTS 0.5% |
| 1% | 4% | 4 | 5% | CTS 1% |
| 1.4% | 5.6% | 4 | 7% | CTS 1.4% |
| 1.9% | 7.6% | 4 | 9.5% | CTS 1.9% |
| 1.3% | 6.7% | 5 | 8% | R5T8 |

### Example 2

### Lubricating Performance of GA/CTS Coacervate Droplets Slurry

Friction coefficient of the different coacervate compositions of Table 1 was measured by tribology.

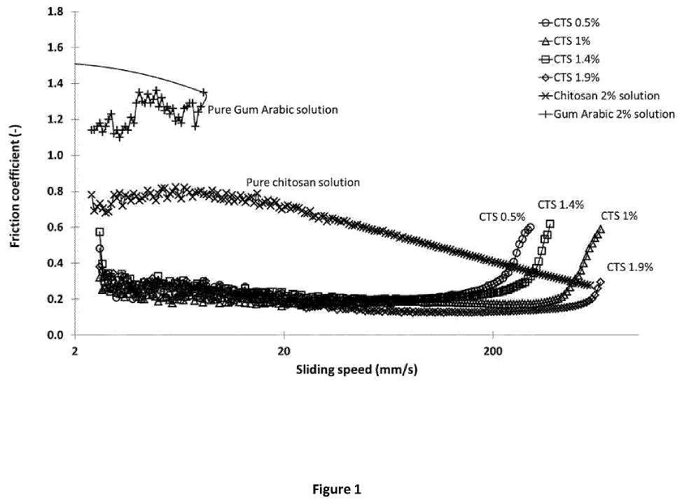

FIG. 1 shows that the combination of the two biopolymers (Gum Arabic and chitosan) enables to decrease significantly the friction coefficient when compared to the individual biopolymers.

### Example 3

### Performance in a Commercial Fat-Free Yoghurt

The commercial fat-free yoghurt Taillefine® 0% from Danone (cow skimmed-milk, powder skimmed-milk, milk proteins, lactobacillius, D vitamin) was used for this example.

### Tribology Results

Different amounts of coacervate phase R5T8 (see composition in table 1) were added in the yoghurt and the effect was measured by tribology.

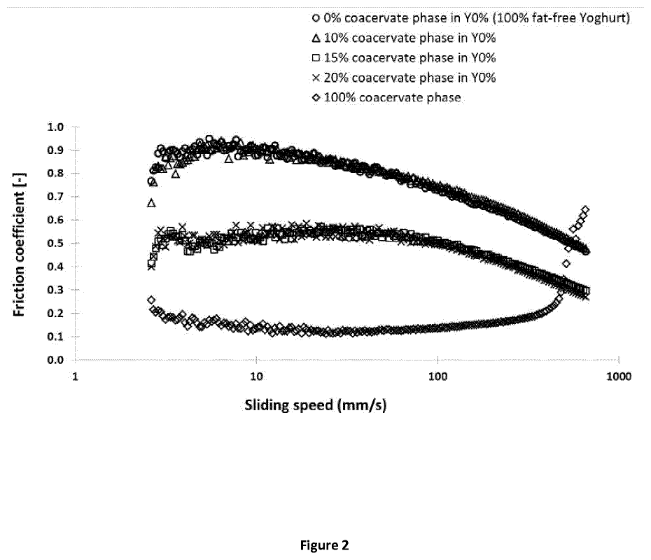

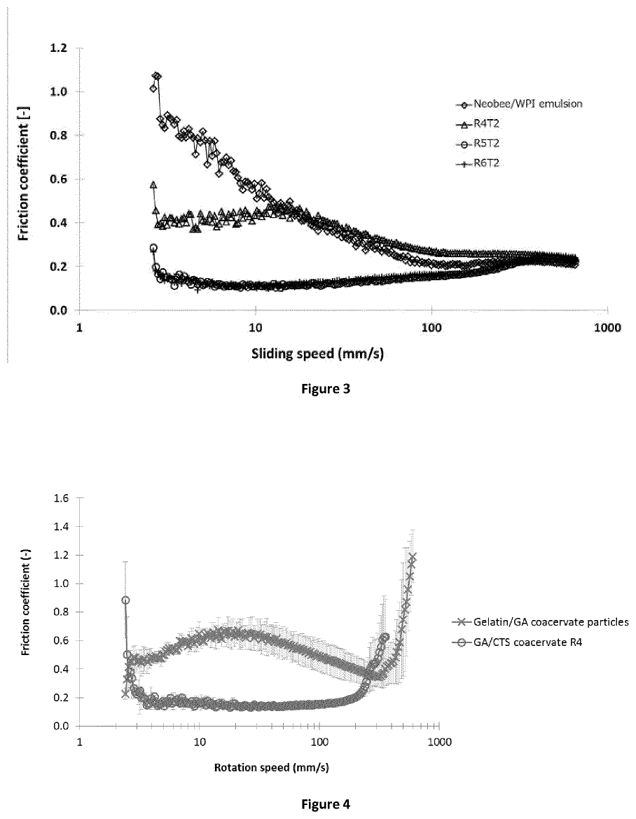

One can see from FIG. 2 that the addition of 15-20% coacervate phase into the fat-free yoghurt enables to decrease the friction coefficient by 33% (15 wt % hydrated coacervate phase corresponds to 3.4 wt % dry GA/CTS coacervate as it contains 77 wt % water).

### Sensory Tests:

A group of 8 panelists were asked to evaluate different sensorial properties of coacervate-containing fat-free yoghurt as compared to the unmodified commercial yoghurt.

After forming the concentrated coacervate phase GA/CTS R5T8, 20 wt % of it was admixed to the fat-free yoghurt Taillefine® 0% (namely 40 g coacervate phase containing 77% water as determined by thermogravimetry+160 g Taillefine 0%).

Panelists found that the coacervate-containing fat-free yoghurt had satisfactory creaminess and thickness. Furthermore, a majority of panelists found that it had higher slipperiness than the commercial product without any coacervate systems.

### Example 4

### Preparation of Complex Coacervate Microcapsules as Defined in the Invention

An O/W emulsion was stabilized with a whey protein isolate (WPI, from Davisco) using an ultra-turrax, before being coated with the coacervate droplets. Medium chain triglyceride (Neobee M5) was used as hydrophobic core material.

53 g of a 2 wt % WPI solution was prepared in 1 wt % acetic acid glacial before adding 8 g of Neobee (medium chain triglyceride) and emulsifying with an ultra-turrax for 1 minute at 24000 rpm (stock emulsion).

The GA/CTS coacervates were prepared separately according to the following compositions:

| | m(CTS2%) | m(GA20%) | m(acetic acid 1%) |
|---|---|---|---|
| R4T2 | 2.00 | 0.80 | 7.20 |
| R5T2 | 1.67 | 0.83 | 7.50 |
| R6T2 | 1.43 | 0.86 | 7.71 |

The coating step was performed by adding 5.5 g of the above-described stock emulsion in the 10-ml coacervate slurry and stirring magnetically for 1 hour.

As a comparison, the lubrication ability of the WPI-stabilized emulsion, not coated with the coacervate was also measured:

The reference emulsion not coated with the coacervate phase is less lubricant than the coated emulsion, which shows a positive effect of the coacervate in the reduction of friction.

Emulsions coated with GA/CTS complexes with ratios R=5 and R=6 have the highest lubrication properties.

### Example 5

### Preparation of Compositions Comprising the Complex Coacervate Defined in the Present Invention

In the following compositions, “hydrated coacervate phase” refers to sample R5T8 and “dry coacervate” refers to sample R5T8 that has been spray-dried.

The following compositions are prepared:

### Composition A

A non-fat ice cream composition is prepared as follows:

1 g dry coacervate
1.6 g fat-free powdered milk
0.1 g pectin
0.2 g thickener (such as guar gum, carrageenan, starch, or India gum)
20 ml of skimmed milk

The skimmed milk is heated until boiling slightly; thereafter, the dry coacervate is added and stirred until slightly thick, followed by addition of the fat-free powdered milk and the pectin. The formulation is then cooled down and the thickener is added while stirring. Flavorings may also be added, such as vanilla, chocolate, or fruit flavoring. Furthermore, one or more sweeteners may be added, such as fructose. Finally, the mixture thus obtained is frozen under shearing in an ice cream freezing machine.

### Composition B

A soft spread formulation with butter-like texture is prepared as follows:
0.6 g dry coacervate
0.8 g fat-free powdered milk
10 ml skim milk
2.0 g of flavoring

The skim milk is heated until just below boiling, and at this point the dry coacervate is added. This mixture is then stirred until thickened. Thereafter, the fat-free powdered milk is added, followed by the addition of the flavoring (for instance, a butter flavoring, or a combination of butter flavoring and savory flavoring or spices).

### Composition C

A fat free salad dressing is prepared as follows:
1 g dry coacervate
2.0 g powdered skim milk
0.06 g carrageenan
20 ml tap water

The ingredients are blended and then heated to just below boiling until the mixture thickens. Then, the mixture thus obtained is cooled down to room temperature. Additionally, flavorings, including fat or cream flavors and spices can be to obtain a salad dressing product with desired flavor profile.

### Composition D

A non-fat chocolate preparation is prepared as follows:

1.2 g dry coacervate
3.0 g powdered skim milk
0.1 g carrageenan
1.0 ml glycerol
20.0 ml tap water;
0.3 g cocoa powder
2.0 g powdered sugar.

The ingredients are mixed and then heated to boiling and until thickened. The mixture is then let to cool down to room temperature, obtaining a spoonable chocolate preparation that may be used either as such, or as a coating or filling.

### Composition E

Fiber-fortified fruit preparations are prepared according to a process comprising mixing sugar, fresh fruit, a fiber source, and optional ingredients. The mixture is heated and cooked at a temperature of 85° C. for a minimum of 30 minutes.

Typical recipes are as follows, with all composition provided in weight-%:

### Swiss Style Fruit Product

40-55% Strawberries or Raspberries
30-40% Sugar
7-12% Fiber Source
1-3% Modified Food Starch
3-5% dry coacervate
Remaining % Water

### Sundae Style Fruit Product

### Strawberry

35-45% Strawberries
15-25% Sugar
15-20% Corn Syrup (63 DE)
5-10% soy fiber
2-4% modified starch
3-5% dry coacervate
Remaining % Water

### Raspberry:

35-45% Raspberries
10-20% Sugar
6-12% Corn Syrup (63 DE)
5-10% soy fiber
1-3% modified starch
3-5% dry coacervate
Remaining % Water

### Composition F

A softening cleansing milk is prepared having the following composition (all ingredients are given in weight-%):

Polyoxyethylene stearate 3%
Glycerin monostearate (Arlacel 165) 3%

### Vaseline oil 36.2%

### Self-emulsifiable PurCellin oil 2%

### Lanolin wax 2%

Carboxyvinyl polymers (Carbopol 941) 0.1%

### Triethanolamine 0.13%

Hydrated coacervate phase 15-20%
Preservative (methyl phydroxybenzoate) 0.3%

### Perfume 0.2%

Demineralized water to complete the mixture to 100% First, the fatty components (polyoxyethylene stearate, glycerin monostearate, Vaseline oil, Pur Cellin oil and lanolin) are blended and melted. This blend is then added to a solution of Carbopol 941, which is obtained by first dissolving the Carbopol in one part of water and then neutralized with triethanolamine. An emulsion of the above components is then formed by mixing the blend of fatty components into the neutralized Carbopol solutions under strong stirring, and the preservative is added. The coacervate phase is carefully dispersed in the rest of the water and added to the emulsion, and the preservative is added.

### Composition G

A hydrating and protective cleansing base is prepared according to the same process as composition F, said base having the following composition in percent by weight:

Stearic acid 2.0%
2,6,10,15,19,23-hexamethyltetracosane (perhydrosqualene) C₃₀H₆₂ 3.5%
Glycerin monostearate (Arlacel 165) 2.0%
Triethanolamine 1.0 Methyl parahydroxybenzoate 0.3%
Carboxyvinyl polymers (Carbopol 941) 0.3%

### Triethanolamine 0.3%

Hydrated coacervate phase 15%

### Perfume 0.3%

Demineralized water to complete the mixture to 100%

### Composition H

A softening cream is prepared according to the same process as composition F, said base having the following composition in percent by weight:

Fatty acid ester (Put Cellin oil) 2.0%

### Vaseline oil 7.0%

Isopropyl myristate 1.5%
2,6,10,15,19,23-hexamethyltetracosane (perhydrosqualene) C₃₀H₆₂ 3.5%

### Lanolin Alcohols (Amerchol L 101) 0.3%

Stearic acid 1.4%
Glycerin monostearate 2.0%
Hexadecylic alcohol 1.0%
Pure cetyl alcohol 0.2%
Preservative (methyl-p-hydroxybenzoate) 0.3%
Carboxyvinyl polymers (Carbopol 941) 0.25%

### Triethanolamine 0.25%

### Perfume 0.2%

### Triethanolamine 0.7%

Hydrated coacervate phase 15%
Demineralized water to complete the mixture to 100%

### Composition I

A day cream is prepared according to the following composition

| Phase | Ingredients | Amounts (%) |
|---|---|---|
| A | Ethoxylated Fatty Alcohol | 5 |
| | Ester ¹⁾ | |
| | Cetyl alcohol | 0.5 |
| | Ceteth-20 (and) Glyceryl | 4 |
| | Stearate (and) PEG-6 Stearate | |
| | (and) | |
| | Steareth-20 ²⁾ | |
| | Squalan ³⁾ | 1 |
| | Paraffin oil | 2 |
| | Petrolatum | 6 |
| B | Water | 55.85% |
| | Hydrated coacervate phase | 20 |
| C | Propylene glycol | 5 |
| | DMDM Hydantoin (and) | 0.15 |
| | Iodopropynyl Butylcarbamate | |
| | 4) | |
| D | Sodium Carbomer | 0.2 |
| E | Perfume | 0.3 |

¹⁾ ARLATONE 985
²⁾ TEFOSE 2561
³⁾ COSBIOL
4) GLYDANT PLUS

Phases A and B are heated separately to 65° C. Phase A is poured slowly into B under vigourous stirring. Then C and D are added.

If needed this base can also be made by hot/cold processing, in which case phase A is heated until homogeneous; phases A and B are heated separately to 65° C.

At 65° C. phase A is added to B, vacuum is applied and the mixture is let to cool down, with additional strong mixing using a colloidal mill for about 15 minutes while the temperature is above 55° C.

Add 50° C., phase C is added and mixed for 5 minutes, followed by addition of D and mixing while cooling down to room temperature until the cream is homogeneous and without lumps. The pH is adjusted to 7 if necessary.

### Example 6

### Comparison of the Lubrication Properties of the Coacervate System of the Invention Versus Gelatin/Gum Arabic Coacervate

Gelatin/gum Arabic coacervate particles were prepared as follows:

Pork gelatin type A (275 Bloom) and gum Arabic (Efficacia®, from CNI) were used as the hydrocolloids to prepare coacervate microbeads.

A stock solution of gelatin (solution A) was prepared by mixing 180 g of warm deionised water and 20 g of gelatine in a vessel until complete dissolution; the solution was then maintained at 50° C.

A stock solution of gum Arabic (solution B) was prepared by mixing 180 g of cold deionised water and 20 g of gum Arabic in a vessel until complete dissolution; the solution was then warmed and kept at 50° C.

24.2 g of solution A was mixed with 24.2 g of solution B in a vessel under gentle agitation (the gelatin/gum Arabic ratio is 1:1). The pH was adjusted to 4.5 with a 50% w/w aqueous lactic acid solution. The system was then diluted by the addition of 112.6 g warm deionised water, which brought the total hydrocolloid concentration to 3% w/w. The mixture was finally cooled to 15° C. at a rate of 0.5° C.min⁻¹. The resulting product finally obtained is a suspension of microbeads of gelatin/gum Arabic coacervate having a mean diameter of 15 um.

A CTS/GA coacervate (R=4 T=5%) was prepared according to our invention as described in Example 1 and Table 1.

Tribology measurements were performed on the concentrated coacervate phases after centrifugation to compare the respective lubrication properties.

As shown in FIG. 4, tribology measurements show that the coacervate prepared according to the invention is up to 6 times more lubricant than the coacervate particles made of gelatin and gum Arabic.

### Example 7

### Performance of the Coacervate Particles of the Invention in a Day Cream

### Preparation of the Coacervate Phase:

The coacervate of composition R5T8 was prepared as follows: 50 g of 20 wt % GA solution prepared in dionized water was admixed to 100 g of CTS 2 wt % solution prepared in 1% lactic acid solution. The mixture was stirred for 30 minutes at room temperature before allowing to sediment. The pH of the slurry is 4.2.

Then, the coacervate suspension was spray-dried on a Mini-Buchi atomizer while stirring to keep the droplets in suspension. A fine white powder was recovered and used for further tests (“Dry coacervate” R5T8).

### Test in Day Cream Application:

3.5 wt % of dry coacervate R5T8 were added in the day cream of Composition I of Example 5. The powder was dispersed in the cream base with the mean of a spatula and the sample was allowed to equilibrate for one night at room temperature.

The day after, 6 non-trained panelists were asked to apply the coacervate-containing cream and the standard cream base (without any introduction of coacervates) on the back of each hand by circular movements. Their comments were collected in the Table below:

| | |
|---|---|
| Coacervate-containing day cream | Reference day cream base |

During application

| | |
|---|---|
| More oily | More thick |
| More fluid | |
| More slippery | |
| More hydrating | |
| Less penetrating | |

After application

Film formation on the skin
Better protection
Smoother aspect of the skin

The day cream containing the GA/CTS coacervate of the invention was preferred over the reference. Moreover, no difference in smell was observed.

1. A method of using a complex coacervate system, the method comprising using the complex coacervate system as a lubricating agent in a consumer product, said complex coacervate system comprising a first biopolymer and a second biopolymer, wherein the first biopolymer is Gum Arabic and wherein the second biopolymer is chitosan.

2. The method according to claim 1, wherein the complex coacervate system is a complex coacervate droplets slurry comprising at least one complex coacervate droplet made of the first biopolymer and the second biopolymer.

3. The method according to claim 1, wherein the complex coacervate system is a complex coacervate microcapsules slurry comprising at least one microcapsule having an oil-based core comprising a hydrophobic active ingredient and a coacervate shell made of the first biopolymer and the second biopolymer.

4. The method according to claim 2, wherein the complex coacervate droplets slurry is obtainable by a process comprising the step of mixing a first and second biopolymer in an aqueous vehicle under conditions sufficient to form a suspension of complex coacervate droplets, wherein this step is performed under acidic conditions.

5. The method according to claim 3, wherein the complex coacervate microcapsule slurry is obtainable by a process comprising the steps of:
(i) mixing a first and second biopolymer in an aqueous vehicle under conditions sufficient to form a suspension of complex coacervate droplets, wherein this step is performed under acidic conditions, and
(ii) adding a hydrophobic core material to the complex coacervate droplets to form core/shell capsules each containing the core material encapsulated by a coacervate shell made of chitosan and Gum Arabic.

6. The method according to claim 1, wherein the weight ratio between gum arabic and chitosan is comprised between 3 and 8.

7. The method according to claim 4, wherein the step of mixing a first and second biopolymer is carried out at a pH between 2.5 and 5.

8. The method according to claim 1, wherein the lubricating agent is a fat replacer.

9. The method according to claim 1, wherein the consumer product is a flavoured or a fragranced product.

10. The method according to claim 9, wherein the flavoured product is chosen from the group consisting of yoghurts and other fermented milk products, milk drinks, and cheese products, sauces, and dressings.

11. The consumer product comprising the complex coacervate system as defined in claim 1.

12. A flavoured product or a fragranced product comprising the complex coacervate system obtainable by a process as defined in claim 4.

13. The flavoured product according to claim 12, wherein it comprises between 0.1 and 20% by weight of the complex coacervate system based on the total weight of the product.

14. The method according to claim 1, wherein the complex coacervate system is a complex coacervate microcapsule slurry comprising at least one microcapsule having an oil-based core comprising a hydrophobic active ingredient comprising a flavor or perfume, and a coacervate shell made of the first biopolymer and the second biopolymer.

15. The method according to claim 5 wherein the hydrophobic core material is flavor or perfume.

16. The flavoured product according to claim 12 wherein the product is in the form of yoghurts, milk drinks, and cheese products, sauces, and dressings.
